(19) United States
(12) Patent Application Publication
(10) Pub. No.: US 2006/0030727 A1
(43) Pub. Date: Feb. 9, 2006

(54) Method and device for the continuous pre-polycondensation of esterification/transesterification products

(75) Inventors: Fritz Wilhelm, Karben (DE); Michael Reisen, Frankfurt am Main (DE)

Correspondence Address: THE FIRM OF KARL F ROSS, 5676 RIVERDALE AVENUE, PO BOX 900, RIVERDALE (BRONX), NY, 10471-0900, US

(73) Assignee: ZIMMER AKTIENGESELLSCHAFT

(21) Appl. No.: 11/198,872

(22) Filed: Aug. 6, 2005

(30) Foreign Application Priority Data
Aug. 7, 2004 (DE) 102004038466.5

(51) Int. Cl. C07C 69/76; B01J 8/04

(52) U.S. Cl. 560/76; 422/188

(57) ABSTRACT

In a method for the continuous pre-polycondensation of esterification/transesterification products the stream freely passes from top to bottom in a vertical reaction apparatus through a plurality of attached, heatable, sloped open-above bases, through product overflow channels connected with each other. In order to be able to set a determined, efficient as possible product level in the channels, the esterification/transesterification products are fed though closed, annular-type, concentric annular channels or through parallel channels, wherein a partial quantity of the product stream flowing in the channel is discharged at the product overflows and the remaining product stream is passed via drainage openings.

### FIELD OF THE INVENTION

Our present invention to a method and an apparatus for the continuous pre-polycondensation of the esterification/transesterification products produced by means of esterification/transesterification of dicarboxylic acids, preferably terephthalic acid or esters of dicarboxylic acids with diols, preferably ethylene glycol, in a vertical reaction apparatus with a plurality of heatable, dead-space free and residue-free channels arranged one below the other, connected at the edge with the wall of the reaction apparatus, opposite bases sloped to the horizontal, with the horizontal, open-on-top, product overflows connected with each other by automatically emptiable drainage openings, with level isobaths, through which the dosed-in esterification/transesterification products flow freely from top to bottom.

### BACKGROUND OF THE INVENTION

A method is known from German Patent 10246251 for the continuous manufacture of polyesters by esterification/transesterification of dicarboxylic acids, preferably terephthalic acid, or esters of dicarboxylic acid, with diols, preferably ethylene glycol.

In that connection for pre-polycondensation, the esterification/transeserification product is fed for the pre-polycondensation to a vertical reactor in which a pressure of 10 to 40% of the diol equilibrium pressure of the pre-polycondensation product discharged from the reactor prevails. The feedstock is fed freely one after another initially through at least one ring-shaped channel reaction zone accompanied by limited heating. It then passes into a radially outer or radially inner annular channel and fed to at least one of a divided ring-shaped channel, in a plurality of concentric annular channels forming a second reaction zone.

It is then conducted one after the other through the annular channels to the outlet, and brought into an agitated sump of the reactor forming a third reaction zone. Subsequently the prepolycondensation product is fed to a polycondensation stage, comprised of at least one horizontal finisher. As a result of the pre-polycondensation carried out in the reactor an increase of the viscosity of the pre-polycondensation product is achieved with comparatively low process temperatures and low pressure.

### OBJECT OF THE INVENTION

It is the object of the present invention to develop the method described above as well as the apparatus for carrying out the method as efficient as possible. This is necessary to achieve defined residence times and the coverage of the heat register located in the channels. Likewise, dead-space, freedom from residue and automatic emptying of the channels as well as uniform steam load should be ensured.

### SUMMARY OF THE INVENTION

This object is attained in accordance with the invention in that the esterification/transesterification products are fed through one or a plurality of closed ring-like concentric annular channels on conical, or pyramidal polygonal bases, or arranged on level bases in at least two opposing, partly level sloped bases and at the product overflows a portion of the product stream flowing in the channel drains off and the remaining product stream is discharged via the drainage openings.

In an especially simple embodiment of the invention the product stream is conducted out of the annular channel in the head region of the reaction apparatus via at least one product overflow and through at least one drainage opening directly into the stirred reaction zone.

For carrying out the method at the product overflows it is advantageous to discharge in each case at least 25 volume %, preferably 50 to 80 volume % of the product stream. In this manner there is a constant level of product in each individual channel. For carrying out the method, in addition to a desired change of throughput of the product stream dosed into the reaction apparatus per unit of time, also unexpected process fluctuations should not influence the quality of the finished product. While it is conceivable at constant throughput to discharge the total product stream through the drainage openings, too large dimensioned drainage openings at lower throughput lead to a lower product level and therewith to uncontrolled holding times and irreproducible reaction results. On the other hand too small drainage openings would lengthen the time needed for complete emptying of the channels.

In the reaction chamber essentially the same pressure of 5 to 100 mbar is suitably maintained above all the channels, while the free space between the channels is sized such that between the exhaust vapor line and the exhaust vapor spaces over the bases and above the sump there is no appreciable loss of pressure.

At their deepest point the channels have in the case of sloped bases at one channel wall or between two channel walls, an isobath. The drainage openings are preferably so positioned and configured that in each case at an end position along the isobath of a channel 5 75 volume %, preferably 20 to 50 volume % of the product streams are discharged.

Since it has been established that in the product stream when flowing through the upper open trough-like channels, a rate profile develops with a slower edge flow at the channel walls and an accelerated flow in the center and as a result of that product discolorations develop at the base and sidewalls of the channels as well as irregular properties at the surface and base-side layer of the product stream, in each channel the central flow is slowed down at least once and the flow at the edges is accelerated at least once.

As a general rule in the method in accordance with the invention the level of product flow in the channels of a base is held essentially constant. A special embodiment of the method in accordance with the invention comprises decreasing the level of the product stream in the channels from base to base or from channel to channel and the total pressure at the channel bases falls below the local equilibrium pressure of the cleaved diol by ≧25%, preferably 50 to 90%.

In order to ensure uniform heating and to avoid a sudden diol evaporation as well as to avoid the foaming and spraying related thereto, the product stream flowing through the upper annular channel attached to the base located in the head region of the reaction apparatus fixed annular channel is heated at a rate of S 0.5 K/min, preferably ≦0.3 K/min, heats. Thereby lo unwanted local steam charge spikes are avoided.

A comparative moderation of the steam loading of the entire system can be achieved according to another feature of the invention in that the product stream flowing out of the annular channel of the attached base in the head region of the reaction apparatus one upper channel and in-flowing product stream in at least one upper channel of the subsequent base divides at least once into two equal oppositely flowing product streams, the partial product streams are fed in each case through half the length of the channels, conducted up until the particular product overflow and are combined at the total product overflow of the subsequent channel.

A further possibility for comparative moderation of the steam load can occur in the way that especially in a product stream flowing through concentrically arranged annular channels, the product stream fed through outer located annular channels is conducted counter to that in the subsequent inner annular channels.

In the device for carrying out the method in accordance with the invention at least one base occupied by at least one annular channel is provided in the head of the reaction apparatus into which the esterification/transesterification product can be fed.

The annular channels can be round or consist of straight pieces, wherein the latter embodiment is simpler to fabricate.

The product overflows consist of straight weirs or piping. A product overflow pipe is formed from either a standpipe, from a swan neck type, with the siphon connected at its upper peak to the exhaust vapor space, or from a discharging standing pipe with an open downstream drain. The product underfloor weirs comprise straight weirs or in each case an uptake enclosing the product overflow pipe.

The drainage openings can be simple openings in down-spouts or dividing walls or at the channel base or are at the deepest point of a swan-neck type siphon's outgoing bypass line. Other arrangements are also possible, insofar as their out-flowing product is taken directly from the base of the channel.

In a preferred embodiment at least the upper attached bases in the head region have a product overflow pipe with a drainage opening for delivery of the product into the succeeding channel located below a subsequent sequential base. In this way, the exhaust space above the upper base can be separated by means of a wall from the remaining exhaust space and the exhaust vapor stream loaded with entrained product droplets can be separately discharged.

In a particularly simple embodiment of the invention the product overflow pipe and the drainage opening feed directly into the stirred reaction zone.

For the division of the product stream into two equal quantity streams it is advantageous to arrange for the product overflow to be diametrically opposite the product inlet in the middle of the channel.

The product overflow pipe is as a rule attached at the end of the channel before a final separating wall.

Adjacent channels are in each case connected with at least one product overflow weir located in the intermediate walls of the channels, wherein an underflow weir is preferably connected upstream to the product overflow weir with or without side columns. By means of such an arrangement, between underflow weir and the overflow weir there is a gap in the path through which the product stream taken from the channel bases is fed to the overflow weir. An alternative is instead of the underflow weir to install an uptake leading to the overflow weir. Irregularities in the pre-condensation product manifest themselves especially in different degrees of polymerization or viscosities. A product with higher viscosity has a greater density than a product of lower viscosity and therefore sinks slowly to the base in the channel. If now preferably the product of higher viscosity is discharged from the channel base, the product having lower viscosity remains longer in the channel and is thus polycondensed to higher viscosities, sinks to the channel base and is conducted from there to the overflow weir. In this manner a controlled comparative moderation of the reaction product is achieved.

The underflow weirs can among other ways be practiced in reaction apparatuses having concentrically arranged channels in addition to the function of baffle plates, which conduct the product stream at the end of a channel in such a way to the overflow weir that no dead-spaces remain. For this purpose it is possible for example that the gap between the channel base and the bottom edge of the underflow weir is not held constant over the entire with of the channel, but enlarge towards a channel wall, so that a larger quantity of the product stream can flow there.

In order to achieve a comparative moderation of the product stream at least one diversion element preferably with breakthroughs is located in each channel. The diversion elements in their simplest form possess straight upper and lower edges. Additionally the comparative modification of the product stream is supported by having a saw-tooth or comb-like profile at the edges. It is thereby possible that the product stream passes above and/or below the diversion elements and/or from time to time passes by and/or through the breakthroughs. Diversion elements fabricated from thin sheets cause practically no losses relative to the evaporation surface and the volume of the product stream and thereby no decrease in productivity.

Between the sides and/or under-edges of the underflow weirs and/or the diversion elements and the channel base and/or the gaps existing at the channel wall through which the edge flow of the production stream can flow unhindered, while the underflow weirs or the diversion elements slow down the central flow, so that this is forced to delay underflow through the underflow weir and/or force flow through the breakthroughs of the diversion elements.

Suitably the underflow weirs and the diversion elements extend over 25 to 100% of the height and 15 to 95% of the width of the channel.

According to another feature of the invention the bases of the reaction apparatus are sloped at 0.5° to 8°. The slope of all of the bases can be the same or the slope of one base can be larger than that of the base arranged above it. By means of an increasing slope from base to base a uniform flow of the product stream whose viscosity increases from base to base increases is ensured. The emptying of the channel is also improved thereby.

For a single flow operated from an undivided product stream through streamed reaction apparatus in each case suitably located for downward streaming, through a dividing wall formed end of channel in the adjoining intermediate wall a conducting overflow weir is provided for none of the product streams at the beginning of the channel sequence.

For a double flow operated from two equal product partial amount product streams through streamed reaction apparatus with central operation and branching of the product stream, with the bases alternating in pairs, the single channel wall at the end and the subsequent channel wall in the middle have an overflow weir joined to them. In such an embodiment it is advantageous to provide for the last channel wall to be closed and as drain for the combined partial streams to provide a connected overflow element in the base of the last channel.

### BRIEF DESCRIPTION OF THE DRAWING

The above and other objects, features, and advantages will become more readily apparent from the following description, reference being made to the accompanying drawing in which:

FIG. 1 is a longitudinal (vertical) axial section though a reaction apparatus, according to the invention;

FIG. 2 is a diagrammatic sectional view through the reaction apparatus along line II-II of FIG. 1;

FIG. 3 is a longitudinal vertical axial section through another reaction apparatus according to the invention;

FIG. 4 is a horizontal section along the line IV-IV of FIG. 3;

FIG. 5 is a sectional view similar to FIG. 2 of a sequential mountable base in a reaction apparatus in accordance with FIG. 1;

FIG. 6 is a sectional view similar to FIG. 4 of a sequential mountable base in a reaction apparatus in accordance with FIG. 3;

FIG. 7 is another sectional view similar to FIG. 4 of a sequential mountable base in the reaction apparatus in accordance with FIG. 3;

FIG. 8 is a partial longitudinal section through a reaction apparatus in the region of a sequential base;

FIG. 9 is a transverse sectional view of a mountable sequential base in a reaction apparatus in accordance with FIG. 8;

FIG. 10 is a longitudinal vertical section through another reaction apparatus in accordance with the invention;

FIG. 11 is a-diagrammatic cross sectional view showing a partition between two successive paths of a channel with an overflow weir and a drainage opening;

FIG. 12 is a cross sectional view showing an underflow weir in greater detail;

FIG. 13 is a cross sectional view in the direction of flow through one of the flow paths showing an underflow weir;

FIG. 14 is a cross sectional view similar to FIG. 13 showing a combined overflow and underflow weir;

FIG. 15 is a cross sectional view showing a congestion weir formed with holes;

FIG. 16 is a cross sectional view illustrating another congestion weir and has beneath it a top view showing the orientation of that weir with respect to the flow direction;

FIG. 17 is a cross sectional view of an embodiment of the underflow weir;

FIG. 18 is a cross sectional view showing an overflow pipe; and

FIG. 19 is a cross sectional view with another illustration of the overflow pipe.

### SPECIFIC DESCRIPTION

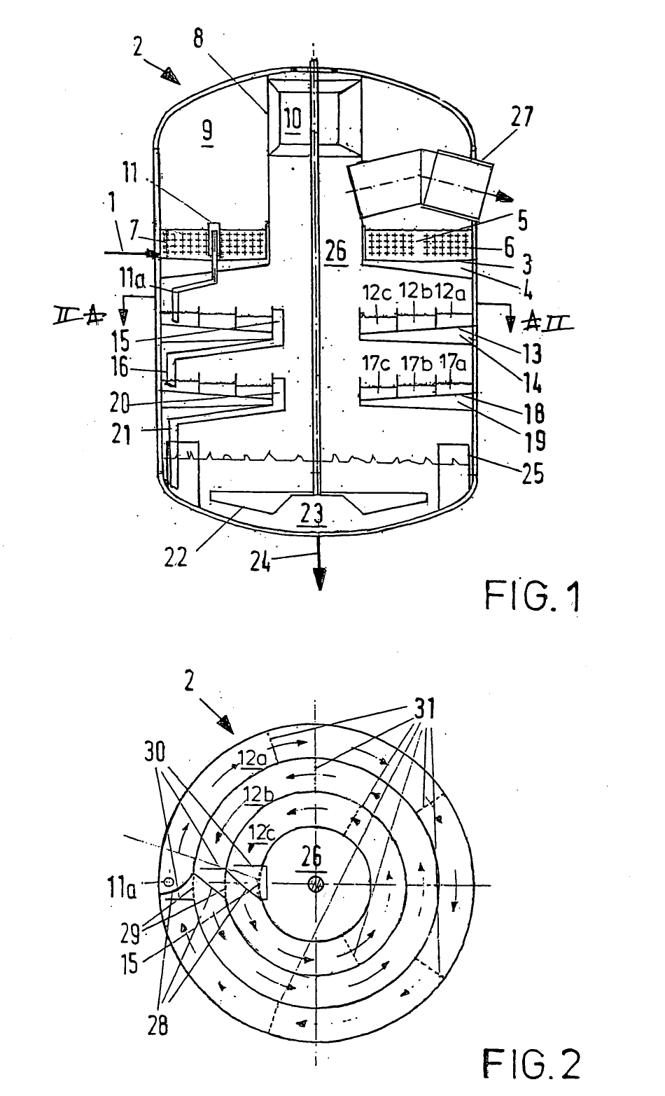

To the apparatus of FIG. 1 and FIG. 2, the esterification product is fed via line 1 to the base 3 located in the head region of the reaction apparatus 2 on one of two oppositely arranged partial cones sloped at 2° with in each case a heat pocket 4 and concentric annular channel 5, in which a heat register from concentric heat pipes 6 is immersed. The esterification product feeds behind a beginning and end 5 forming vessel wall 7 of the annular channel 5.

Above annular channel 5 is located the circulating separating wall 8, enclosing an exhaust vapor space 9 between the inner wall 8 of annular channel 5 and the inside of the housing of the reaction apparatus 2.

On the housing side of the dividing wall 8 a cyclone-type separation device 10 can be attached, by means of which entrained product droplets are separated from the vapors. At an end of annular channel 5 ahead of the vessel wall 7 and in the concentric isobath formed by the annular channel 5 in a riser 11 extending upwardly from an overflow pipe 11a and covered by a downwardly open cup.

The reaction product after running through annular channel 5 passes into this cup and down through riser 11 and overflow pipe 11a into the radially outer lying annular channel of the three annular channels 12a, 12b, 12c sloped at about 4° toward the middle of the container.

The channels have a sloped sequential base 13 with heat pocket 14.

After flowing through annular channels 12a-12c the reaction product flows from the radially inner lying annular channel 12c over the product overflow weir 15 via line 16 into the outer lying annular channel 17a. The latter is one of three annular channels 17a, 17b, 17c inclined at about 4° toward the middle of the container by virtue of a sloped sequential base 18 with heat pocket 19.

The reaction product runs out from the radial inner lying annular channel 17c over a product overflow weir 20 through line 21 into the level-regulated stirred sump 23.

The sump is provided with an impeller 22 having a vertical driveshaft. From there, the product is fed via line 24 attached in the base of the reaction apparatus 2 to a not-shown polycondensation stage.

In the sump region of the reaction apparatus 2 baffle deflector plates 25 are attached, which strengthen the surface renewal and the polycondensation output in the sump 23. The exhaust vapors arising in annular channels 12a-c, 17a-c of the sequential bases 13, 18 and in sump 23 are conducted internally to the top through the chimney 26 formed by sequential bases 13, 18 and the base 3, combined with the exhaust vapors from the separation device 10 and the attached exhaust vapor inlet 27 and conducted out of the reaction apparatus 2.

The beginning and end of the annular channels 12a-12c, 17a-17c on the sequential bases 13, 18 are in accordance with FIG. 2 in each case determined through a chamber wall 28, wherein at the ends of the ring channels 12a-12c, 17a-17c a product overflow weir 15, 29 with upstream underflow weir is provided. In the annular channels 12a-12c, 17a-17c, congestion elements 31 are attached.

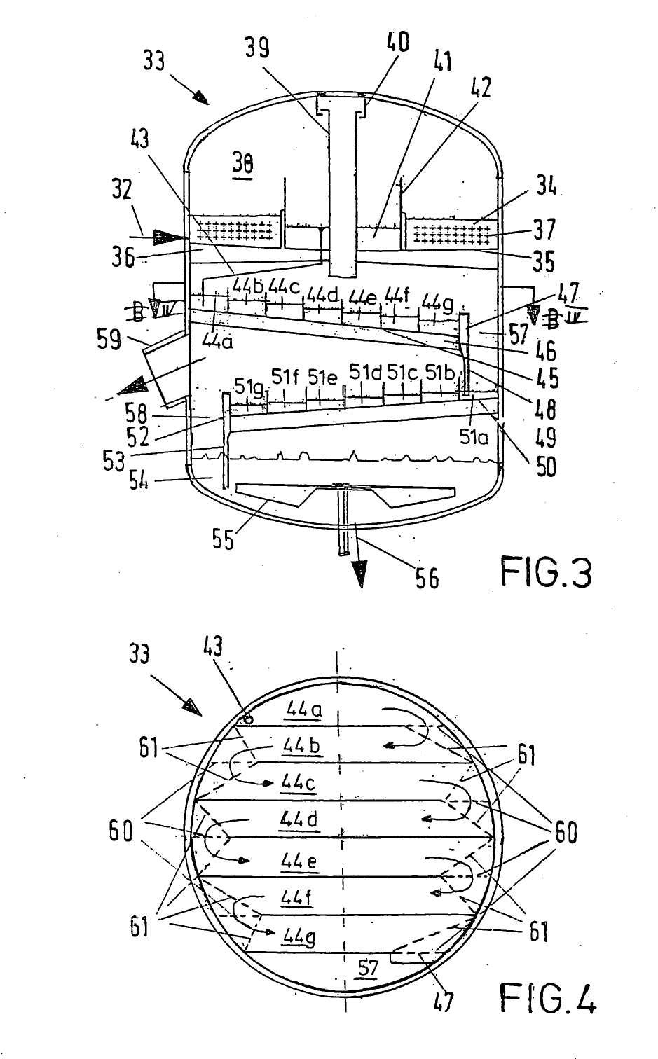

In accordance with FIG. 3 and FIG. 4, the esterification product is fed into the apparatus via line 32 in which in the head region of the reaction apparatus 33 a concentrically arranged annular channel 34 is provided on which is attached a conical base 35 sloping toward the center of the reaction apparatus 33 with its heat pocket 36.

In the annular channel 34 a heat register is arranged in the form of a coil concentric turns of heating pipe 37. The base 35 has a central opening in which a downcomer 39 extends downwardly through the annular channel 34 from the exhaust vapor space 38 into which the exhaust vapor discharges.

Between the upper end of the downcomer 39 and the housing of the reaction apparatus 33 a trap 40 is located for separation of entrained reaction product droplets from the exhaust vapors.

After passing through channel 34 the reaction product enters the channel space 41 existing between the inside wall of annular channel 34 and the downcomer 39 which is surrounded by a cylindrical protective partition 42 and from which the reaction product after suitable passage through the overflow pipe 43 enters the upper channel 44a of a plurality of parallel channels 44a, 44b, 44c, 44d, 44e, 44f, 44g which are attached to a downwardly sloping sequential base 49 formed with pipes with heat pocket 46.

After flowing along parallel channels 44a-44g the reaction product passes over the attached overflow weir 47 via the pipe 48 into the upper parallel channel 51a of descending second sequential base 49 with heat pocket 50 via attached parallel channels 51a, 51b, 51c, 51d, 51e, 51f, 51g.

From the lower parallel channel 51g of the sequential base 49 the product passes via an overflow weir 52 in the outer wall of the lower parallel channel 51g and the pipe 53 into the level-controlled sump 54, which is agitated by means of a vertical drive shaft and its impeller 55.

The product is fed further via line 56 to a not-shown polycondensation stage.

Between the outer wall of the lower parallel channel 44g, 51g of the sequential bases 45, 49 in each case and the oppositely lying wall of the reaction apparatus 33, the sequential bases 45, 49 have a circular section shaped gap for the passage of the exhaust vapors formed, which are discharged via an exhaust vapor line 59 in the lower section of the reaction apparatus 33 to the exterior. The intermediate walls existing between the parallel channels 44a-44g, 51a-51g in each case have between the channel ends and the beginning of the sequential channels an overflow weir 60 to which in each case an overflow weir 61 with lateral openings is connected up and/or downstream.

Numerous variations of the described reaction apparatus 2, 33 are possible. Thus for example instead of an impeller agitator, a horizontally arranged cascade agitator with horizontal drive shafts can be employed.

For the sequential base 62 represented in FIG. 5 with three annular channels 63 the reaction product flowing in via pipe 11a in the radial outer first annular channel is branched into two equal partial product streams and the partial product streams are piped in each case through half the length of the annular channel up to a product overflow weir 64, recombined there and fed over to the second annular channel. There the product stream is again branched into two equal partial product streams which are in each case fed through half the length of the annular channel up to the nearest product overflow weir 66, re-combined and fed into the radial inner lying annular channel. Behind the product overflow weir 65 the combined product stream is branched again into two partial product streams which in each case flow through the half the length of the radially lying inner annular channel up to a product overflow weir 66, are re-combined there and the product stream is fed to a further reaction zone not shown here. Downstream of the product overflow weirs 64, 65 product underflow weirs 67 are connected up and/or downstream. The branched partial product streams in the radial outer lying annular channel in each case pass after completion of the branching over a product underflow weir 68.

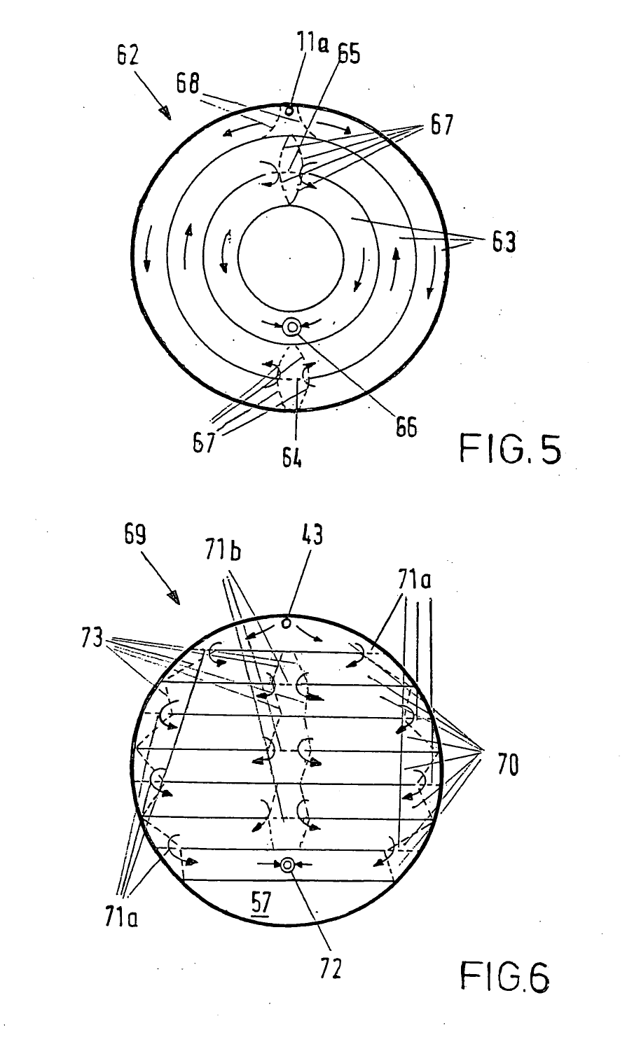

FIG. 6 represents a sequential base 69 with eight parallel channels 70 with, attached in the intermediate walls, product overflow weirs 71a alternating in pairs at the outer channel ends and central product overflow weirs 71b in the particular sequential walls with the exception of the last lower parallel channel. Instead of a central product overflow weir in the base of the last lower parallel channel, a riser is provided.

For the feed the overflow pipe 43 in the upper first parallel channel supplies the product stream which branches into two equal oppositely running product streams, which after passing product overflow weir 71a in the intermediate wall at the outer ends of the parallel channel in the second subsequent parallel channel are reversed and again recombined in the center of the channel base.

The entire product stream passes the central product overflow weir 71b to the third, and subsequently to the fifth and seventh parallel channel or after repeated branching into partial product streams, in the transition the edge located product overflow weirs 71a to the fourth, sixth and eighth parallel channel. Therefore exactly half of the product amount goes through the parallel channels over half the channel length.

The total product flow is discharged via the overflow pipe 72 that is arranged in the base of the last lower parallel channel. Between the outer wall of the last lower parallel channel and the oppositely lying wall of the reaction apparatus there is an opening for passage of exhaust vapors 57 in the form of a circular section. The overflow weirs 71a, 71b are connected upstream and/or downstream to underflow weir 73.

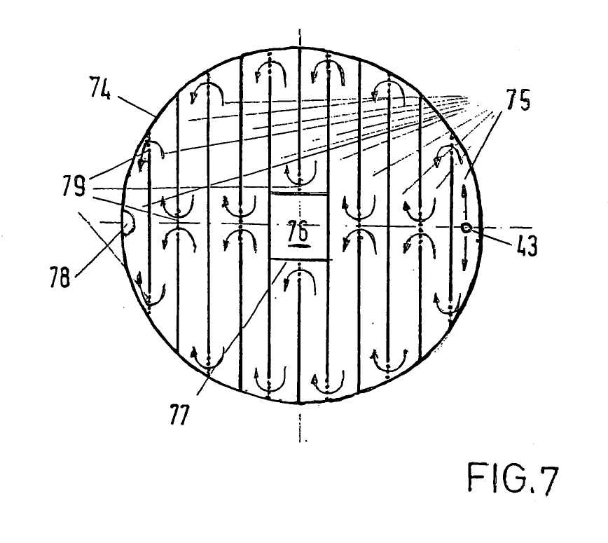

Twelve parallel channels 75 are arranged on the sequential base 74 represented in FIG. 7, wherein the product stream given up in the center of the upper first parallel channel is divided into two equal partial product streams. The central passage opening 76 for the exhaust vapors consists of an attached rectangular chimney 77 in the region of the sixth and seventh parallel channel.

At the same time the wall or casing of the reaction apparatus forms the outer wall of the last lower parallel channel. At its deepest point an approximately semicircular-shaped drain line 78 is attached as overflow for carrying away the product stream from the last lower parallel channel into the first upper parallel channel of another sequential base below the first and not shown here.

The intermediate walls of the parallel channels 75 possess, beginning as in FIG. 6 with the upper first parallel channel alternating at the ends and in the center in each case, a respective product overflow weir 79, wherein as a result of the arrangement of the chimney 77 for removal of the exhaust vapors, the intermediate wall between the sixth and seventh discontinuous parallel channel and in which at the chimney 77 adjacent ends of the intermediate wall sections in each case form a product overflow weir 79 with the half width located in the middle of an intermediate wall.

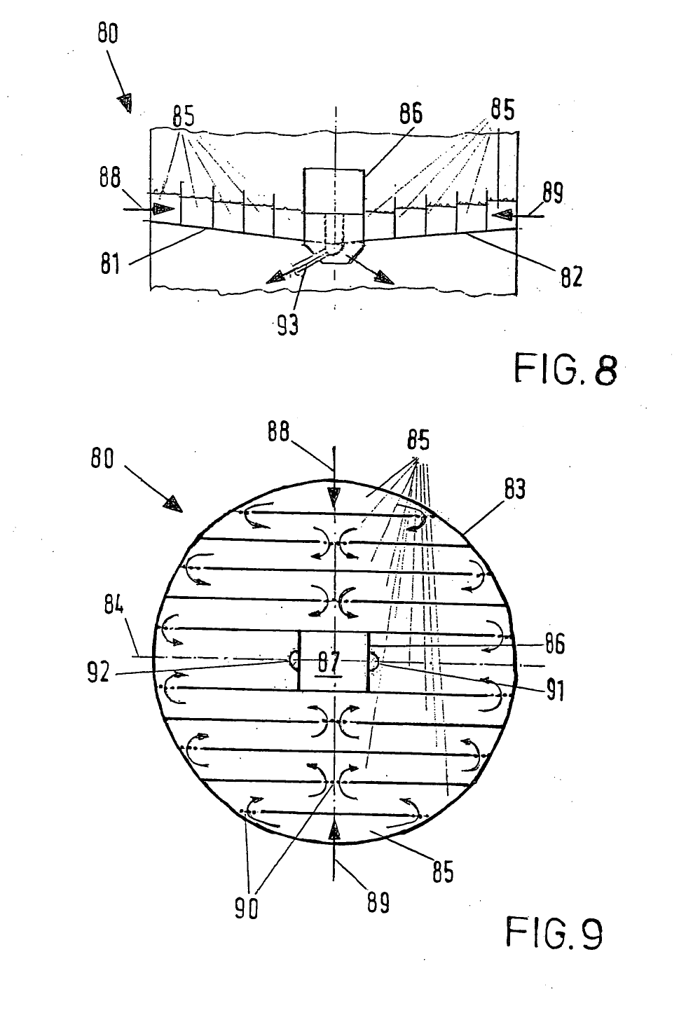

In accordance with FIG. 8 and FIG. 9 in the reaction apparatus 80 a sequential base 83 consisting of one of two sloped base sections 81, 82 descending opposite each other is employed on which eleven channels running parallel to the parallel channels 85 are arranged horizontally in the vertical center plane 84 of the reaction apparatus 80. In the central region of the sequential base 83 a breakthrough 78 surrounding a chimney 86 is located for discharge of the exhaust vapors. A product stream is conducted to the upper first parallel channel of the particular base section 81, 82 via a central feed 88, 89 and in each case branched into two equal product streams at the ends and in the middle of the intermediate walls of the parallel channels 85, in each case an overflow weir 90a, 90b is attached. On both sides of the perpendicular center plane of the reaction apparatus 80 at the chimney 86 ending parallel channel sections, the product streams flow together and are in each case fed via attached discharge lines 91, 92 at the channel ends of the chimney 86 in the region of the isobath 84, via in each case a connection line 93 to a not-shown sequential base.

On the sequential bases 74, 83 in accordance with FIG. 7 and FIG. 9 upstream and downstream weirs are provided analogous to the sequential bases in accordance with FIG. 6, however not shown here.

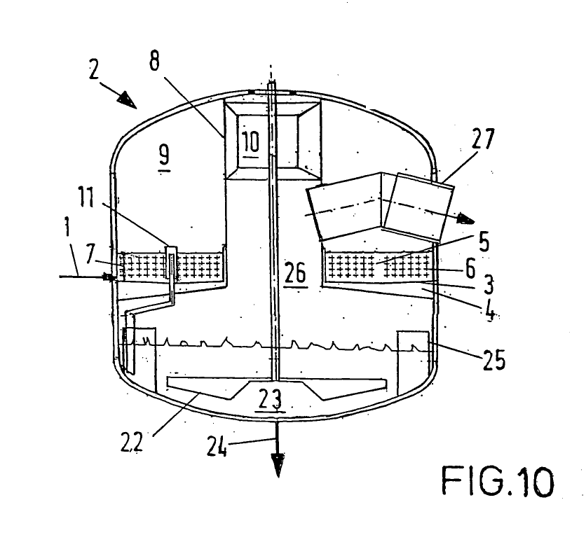

In accordance with FIG. 10, in a particularly simple embodiment of the invention, the esterification product is fed in via line 1 to the concentric annular channel 5 on base 3 located in the head of the reaction apparatus 2 on one of two, with in each case partial, cones sloped about 2° to each other and arranged with a heat pocket 4. In the concentric annular channel 5, concentric heat pipes 8 form a heat register. Above the annular channel 5 is located the dividing wall 8. The vapor passes through a closed-off exhaust vapor space between the inner wall 8 of channel 5 and the inner side of the shell or casing of the reaction apparatus 2. In the casing-side section of the dividing wall 8 a separation device 10 is mounted, by means of which droplets carried over from the exhaust vapors are separated. Via the overflow pipe rising upwards in the riser 11 as well as the drainage opening, not shown in FIG. 10, the reaction product after passing through annular channel 5 is fed downwards via the extension of the overflow pipe into the level controlled sump 23. The latter has an impeller 22 with perpendicular drive shaft and from there the product is fed further via an attached line 24 in the base of reaction apparatus 2 to a polycondensation stage, not shown here.

The exhaust vapors arising in sump 23 are conducted through the chimney 26 formed from the base 3 into the reaction apparatus 2 above, combined with the exhaust vapors coming from the separation device 10, and through the exhaust vapor line 27 attached in the head region of the reaction apparatus 2, and conducted out of the reaction apparatus 2. The arrangement of the overflow pipe with riser and drainage opening preferably corresponds to the embodiment represented by FIG. 18. Principally however the overflow devices shown in FIG. 17 and FIG. 19 are suitable.

Suitable illustrations of features of the method in accordance with the invention are found in FIG. 11 through FIG. 19, which show:

FIG. 11 shows a front elevation in the partition 94 of two adjacent reaction product flow-through channels of an overflow weir 95 mounted with a saw-tooth type formed overflow edge 86 and with a drainage opening 97 in the rearmost dead corner in the chamber sheet.

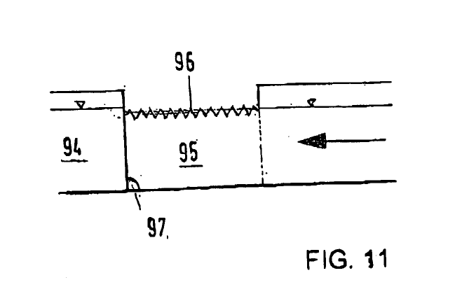

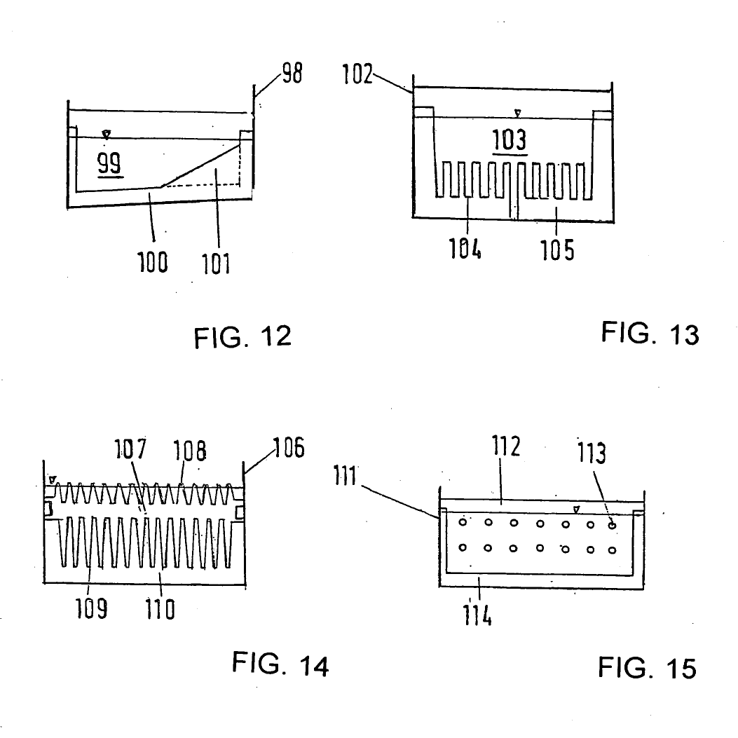

In FIG. 12 an attached underflow weir 99 is seen in a reaction product flow-through channel 98, that forms a gap 100 with the sidewalls and the base of channel 88, which is widened in the region of a corner by means of a wedge-shaped recess 101 of the underflow weir.

FIG. 13 is a front view of an underflow weir 103 employed in one of a reaction product flow-through channel 102 with comb-like lower edge 104 that forms an edge-gap 105 with the sidewalls and the base of channel.

FIG. 14 shows a congestion weir 107 arranged in a reaction product flow-through channel 106, whose top edges 108 are saw-tooth like and whose lower edges 109 are comb-like. Between the lower edges and the sidewalls and base of the channel 106 there is a gap 110.

FIG. 15 shows a congestion weir 112 mounted in one of a reaction product flow-through channel 111 with holes 113 which form an edge-gap 114 with the walls and the base of channel 111.

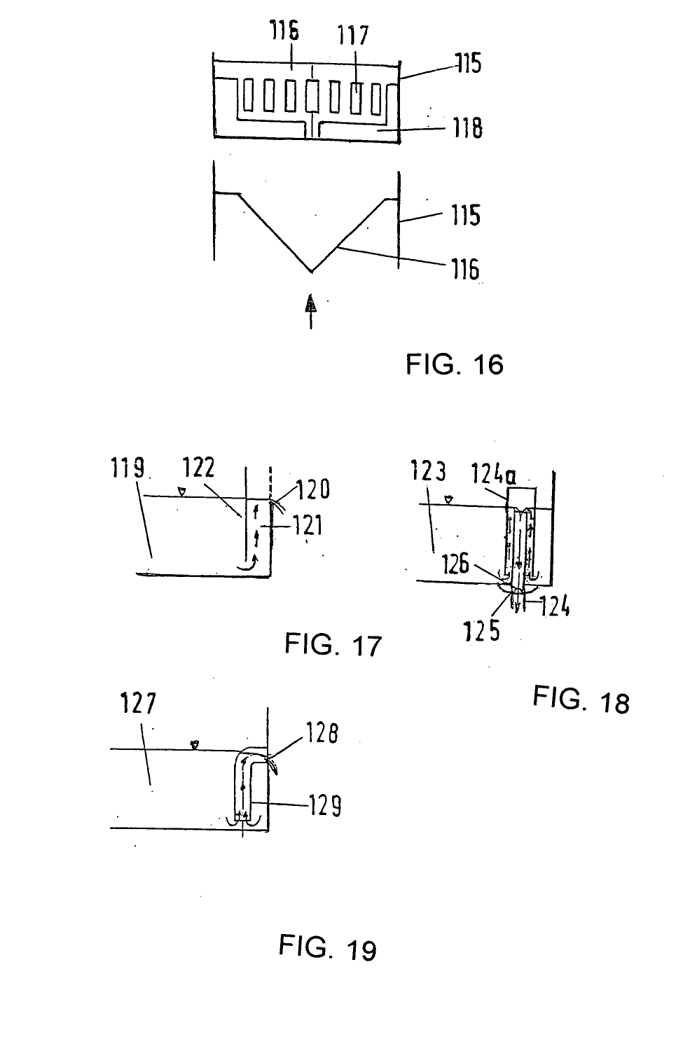

FIG. 16 shows a V-shaped congestion weir 116 employed in the channel 115, whose peak is pointed counter to the flow direction of the reaction product stream in the channel 115. The congestion weir 116 possesses slot-like breakthroughs 117 and forms a gap 118 with the sidewalls and the base of channel 115.

FIG. 17 shows in the wall at the ends of one of a reaction product flow-through channel 119 attached product overflow weir 120, to which by means of formation of an upstream slit 121 a product underflow weir 122 is connected upstream, so that reaction product conducted to the product overflow weir 120 is discharged from the base of channel 119.

In accordance with FIG. 18, at the end of a reaction product flow-through channel 123 an overflow pipe 124 with vertical riser 124a is employed in the base, through which the reaction product is discharged from the base of channel 123. The base of channel 123 in the region of the overflow pipe 124a is provided with a depression 125, so that in the case of an emptying of channel 123 its drainage is ensured via an attached breakthrough 126 in the overflow pipe 124a, of the height of the depression 125.

In FIG. 19 in the wall at the end of a reaction product flow-through channel 127 from a product overflow weir 128 is attached, to which the reaction product discharged from the base of channel 127 is fed in through a riser 129.

We claim:

1. A method of carrying out a continuous pre-polycondensation of an esterification or transesterification product manufactured by the esterification or transesterification of a dicarboxylic acid or ester with a diol which comprises the steps of:
(a) providing in a vertical reaction apparatus, a plurality of bases disposed one above another and which are generally horizontal with a slight downward inclination;
(b) feeding said product into a channel on an upper one of said bases and causing said product to flow along multiple paths of said channel and to overflow from one path into another on said upper one of said bases while said product is heated from below said channel;
(c) discharging said product from said upper one of said bases into a channel formed on a lower one of said bases by overflow;
(d) causing said product to flow along multiple paths of said channel of said lower one of said bases and to overflow from one path into another on said lower one of said bases while said product is heated from below said channel of said lower one of said bases;
(e) collecting gases above said bases in a head of said apparatus; and
(f) discharging a pre-polycondensed product from said apparatus.

2. The method according to claim 1 wherein at least 25 vol % of the product stream discharges at each product overflow.

3. The method according to claim 2 wherein 50-80 vol % of the product stream discharges at each product overflow.

4. The method according to claim 1 wherein in a reaction chamber of said apparatus an essentially equal pressure of 5 to 100 mbar prevails above all the channels.

5. The method according to claim 1 wherein at one end location along an isobath of a channel, ≦75 vol % of the product stream is discharged.

6. The method according to claim 1 wherein in each channel an interior stream of the product stream is slowed down at least once and an edge stream is accelerated.

7. The method according to claim 1 wherein a product stream level in a channel of a base is held essentially constant.

8. The method according to claim 1 wherein a product stream level decreases in the channels from base to base or from channel to channel and a total pressure at the channel bases falls below the local equilibrium pressure of a cleaved diol by ≧25%.

9. The method according to claim 1 wherein a product stream transported through the channel of the base arranged in the head of the reaction apparatus is heated at a rate of ≦0.5 K/min.

10. The method according to claim 1 wherein a product stream flowing out of the annular channel of the base attached in the head of the reaction apparatus and the product stream flowing into an upper channel of the sequential base is divided at least once into two equal oppositely flowing partial product streams and the partial product streams are fed through the half length of the channels up to the particular product overflow and are combined at the common product overflow to a subsequent reaction zone.

11. The method according to claim 1 wherein a product stream flowing at the product overflows is drawn predominantly from the channel bases.

12. The method according to claim 1 wherein a product streams of adjacent annular channels especially the product streams in the outer lying annular channel, and in the inner subsequent annular channels, are feed in opposite directions.

13. The method according to claim 1 wherein the reaction apparatus contains a channel from which the product stream is fed to a subsequent reaction zone.

14. A device for the continuous pre-polycondensation of the esterification/transesterification products produced by esterification/transesterification of dicarboxylic acids, preferably terephthalic acid or esters of the dicarboxylic acids with diols, preferably ethylene glycol in a vertical reaction apparatus having one or a plurality of heatable sloped-to-the-horizontal bases arranged one above the other, connected at the edge with the wall of the reaction apparatus with horizontally, connected with each other by open product overflows automatically emptiable, flow-through channels free of dead space and residue, with horizontal isobath, through which the dosed esterification/transesterification products stream freely from top to bottom, wherein the esterification/transesterification products pass through annular-type closed, concentric annular channels attached on conical or pyramidal polygonal bases, or are fed to level bases or to at least two parallel sloped partially even channels attached to bases and at the product overflows a partial amount of the product stream flowing in the channel discharges and the remaining product stream is discharged via the drainage openings, characterized in that at least the upper attached base in the head region of the reaction apparatus has at least one annular channel, into which the esterification/transesterification product may be fed.

15. The device according to claim 14 wherein the upper attached base in the head region of the annular channel has an overflow pipe as well as a drainage opening for the discharge of product into the sequential channel, or in the subsequent reaction zone located on the underlying sequential base.

16. The device according to claim 14 for feeding the product over two equal, oppositely flowing streams, the product overflow is arranged diametrically opposite to the product inlet, in the middle of the channel.

17. The device according to claim 14 wherein an overflow pipe is arranged at the end of the channel determined by a separating wall.

18. The device according to claim 14 wherein adjacent channels are in each case connected through at least one product overflow weir.

19. The device according claim 14 wherein an underflow weir, with or without side gaps, is connected upstream and/or downstream of the product overflow weir.

20. The device according to claim 14 wherein the upstream underflow weir is attached with formation of a vertical gap before the product overflow weir.

21. The device according to claim 14 wherein the underflow weir consists of a riser.

22. The device according to claim 14 wherein in each channel at least one congestion element is provided with attached breakthroughs.

23. The device according to claim 14 wherein the underflow weir and the congestion element extend over 25 to 100% of the height and 15 to 95% of the width of a channel.

24. The device according to claim 14 wherein the bases of the reaction apparatus are sloped about 0.5 to 8°.

25. The device according to claim 24 wherein the slope of all bases is the same or the slope of a base arranged over it is larger versus the one above it.

26. The device according to claim 14 wherein for the central task, the division of the product stream into two equal partial quantity streams has alternating in pairs over the base which have one channel wall at the end and the subsequent channel wall and a product overflow weir in the middle.

27. The device according to claim 14 wherein the last channel is closed and the discharge for the combined partial stream amounts, comprise one attached overflow element in the base.
